(19) United States
(12) Patent Application Publication
(10) Pub. No.: US 2003/0050700 A1
(43) Pub. Date: Mar. 13, 2003

(54) Vertebral arch spacer

(75) Inventor: Shun-ichi Kihara, Ohtsu (JP)

Correspondence Address: GREENBLUM & BERNSTEIN, P.L.C., 1950 ROLAND CLARKE PLACE, RESTON, VA, 20191, US

(73) Assignee: ASAHI KOGAKU KOGYO KABUSHIKI KAISHA, Tokyo (JP)

(21) Appl. No.: 10/237,748

(22) Filed: Sep. 10, 2002

(30) Foreign Application Priority Data
Sep. 11, 2001 (JP) P2001-275489

(51) Int. Cl. A61F 2/44

(52) U.S. Cl. 623/17.11

(57) ABSTRACT

A vertebral arch spacer (1) according to the invention has an insertion portion (2) to be inserted in a gap formed by opening a vertebral arch (120) toward either side, and an abutment portion (3) capable of abutting on a spinous process (130) cut away from the vertebral arch (120). An abutment surface 31 of the abutment portion (3) is extended rearward beyond an opened portion (123) of the vertebral arch (120), and abuts on the spinous process (130) from the side. The vertebral arch spacer (1) is provided with a through hole (5). A thread (50) is inserted through the through hole (5), a through hole (129) formed on the vertebral arch (120) and a through hole (132) formed on the spinous process (130) and is thus bound. Consequently, the spinous process (130) can be fixed more reliably. The thread (50) can also be inserted through a through hole (128) formed on a portion (124) remaining on the vertebra side and a through hole (4) of the vertebral arch spacer (1) and can be thus bound.

### BACKGROUND OF THE INVENTION

The present invention relates to a vertebral arch spacer, and more particularly to a vertebral arch spacer to be used in a one-sided insertion, open-door type laminoplastic laminotomy.

The one-sided insertion, open-door type laminoplastic laminotomy is conducted as a medical treatment for cervical spondylosis myelopathy, osteosis of the posterior longitudinal ligament or osteosis of yellow ligament.

In the one-sided insertion, open-door type laminoplastic laminotomy, one of sides (either side) of a vertebral arch is cut, and the other side is used as a hinge to open the vertebral arch, thereby expanding a vertebral canal. A vertebral arch spacer is used as an implant material to be inserted in a gap formed by opening the vertebral arch. The use of the vertebral arch spacer forms an expanded vertebral arch.

In such a one-sided insertion, open-door type laminoplastic laminotomy, a spinous process is cut away from the vertebral arch with a nuchal ligament, an on-spine and inter-spine ligament and a musculus remaining attached thereto, and after the vertebral arch is expanded, this spinous process is returned to a center of the vertebral arch so that the cut spinal process is fixed to the expanded vertebral arch by synostosis.

The use of the vertebral arch spacer, however, causes problems in that, since the position of the spinous process cannot be maintained reliably, the spinous process is shifted or the synostosis is delayed due to the flexion and extension of vertebrae after surgery.

### SUMMARY OF THE INVENTION

It is an object of the invention to provide a vertebral arch spacer capable of reliably holding the position of a spinous process cut away from a vertebral arch.

The object can be achieved by the invention according to the following (1) to (20).

(1) A vertebral arch spacer to be inserted into a gap formed by cutting one of sides of a vertebral arch and opening the vertebral arch by using the other side as a hinge portion, comprising:

an insertion portion to be inserted in the gap; and

an abutment portion capable of abutting on a spinous process cut away from the vertebral arch.

Consequently, it is possible to provide the vertebral arch spacer in which the vertebral arch can reliably be expanded and the position of the spinous process cut away can surely be held.

(2) The vertebral arch spacer according to the (1), wherein the abutment portion is formed to be protruded outward from the insertion portion.

Consequently, the position of the spinous process cut away can be held more reliably.

(3) The vertebral arch spacer according to the (1), wherein the abutment portion is protruded rearward from the expanded vertebral arch when the insertion portion is inserted in the gap.

Consequently, the position of the spinous process cut away can be held more reliably.

(4) The vertebral arch spacer according to the (1), wherein the abutment portion has an abutment surface to be substantially parallel with a median plane when the insertion portion is inserted in the gap.

Consequently, the position of the spinous process cut away can be held more reliably.

(5) The vertebral arch spacer according to the (1), wherein the abutment portion can abut on the spinous process laterally.

Consequently, the position of the spinous process cut away can be held more reliably.

(6) The vertebral arch spacer according to the (1), further comprising a first surface facing an inside of the expanded vertebral arch and a second surface facing an outside of the expanded vertebral arch in a state of an insertion in the gap.

Consequently, the vertebral arch can be expanded more reliably.

(7) The vertebral arch spacer according to the (6), wherein the first surface is a curved concave surface.

Consequently, a vertebral canal can be expanded still more.

(8) The vertebral arch spacer according to the (6), wherein the abutment surface capable of abutting on the spinous process and the second surface form an acute angle.

Consequently, it is possible to obtain a shape which is more compatible with a living body.

(9) The vertebral arch spacer according to the (1), further comprising a through hole capable of inserting a fixing member to be used for a fixation to an opened portion of the vertebral arch.

Consequently, the vertebral arch can be expanded more reliably.

(10) The vertebral arch spacer according to the (9), wherein it is possible to fix the spinous process by inserting the fixing member through a through hole formed on the spinous process.

Consequently, the position of the spinous process cut away can be held more reliably.

(11) The vertebral arch spacer according to the (1), further comprising a through hole capable of inserting a fixing member to be used for a fixation to a portion remaining on a vertebral body side of the vertebral arch.

Consequently, the vertebral arch can be expanded more reliably.

(12) The vertebral arch spacer according to the (9), further comprising a groove in which the fixing member can be inserted.

Consequently, the shift and looseness of the fixing member can be prevented more reliably.

(13) The vertebral arch spacer according to the (1), wherein an engagement portion capable of being engaged with a cut portion of the vertebral arch is provided on each of ends of the insertion portion.

Consequently, the vertebral arch can be expanded more reliably.

(14) The vertebral arch spacer according to (13), wherein the engagement portion is constituted by a concave portion or a notched portion in which at least a part of the cut portion can be inserted.

Consequently, the vertebral arch can be expanded more reliably.

(15) The vertebral arch spacer according to the (1), further comprising a portion in which a thickness is gradually decreased from a front side toward a rear side.

Consequently, when a spinal column is bent backward, the vertebral arch spacers provided in upper and lower vertebrae can be prevented more reliably from coming in contact with each other.

(16) The vertebral arch spacer according to the (1), wherein a ceramics material is used as a constitutive material.

Consequently, it is possible to obtain a vertebral arch spacer having an excellent workability.

(17) The vertebral arch spacer according to the (16), wherein the ceramics material comprises a calcium phosphate based compound.

Consequently, it is possible to obtain a vertebral arch spacer having an excellent bioaffinity.

(18) The vertebral arch spacer according to the (17), wherein the calcium phosphate based compound has a Ca/P ratio of 1.0 to 2.0.

Consequently, it is possible to obtain a vertebral arch spacer having a more excellent bioaffinity.

(19) The vertebral arch spacer according to the (17), wherein the calcium phosphate based compound is hydroxyapatite.

Consequently, it is possible to obtain a vertebral arch spacer having a particularly excellent bioaffinity.

(20) The vertebral arch spacer according to the (1), wherein a porosity of the vertebral arch spacer is 0 to 70%.

Consequently, the adhesion of the vertebral arch spacer onto a bone tissue can be promoted.

The present disclosure relates to the subject matter contained in Japanese patent application No. 2001-275489 (filed on Sep. 11, 2001), which is expressly incorporated herein by reference in its entirety.

### BRIEF DESCRIPTION OF THE DRAWINGS

FIG. 1 is a plan view showing an embodiment of a vertebral arch spacer according to the invention,

FIG. 2 is a view seen in a direction of an arrow X in FIG. 1,

FIG. 3 is a view for sequentially explaining a one-sided insertion, open-door type laminoplastic laminotomy using the vertebral arch spacer shown in FIG. 1,

FIG. 4 is a view for sequentially explaining the one-sided insertion, open-door type laminoplastic laminotomy using the vertebral arch spacer shown in FIG. 1,

FIG. 5 is a view for sequentially explaining the one-sided insertion, open-door type laminoplastic laminotomy using the vertebral arch spacer shown in FIG. 1, and

FIG. 6 is a view for sequentially explaining the one-sided insertion, open-door type laminoplastic laminotomy using the vertebral arch spacer shown in FIG. 1.

### DESCRIPTION OF THE PREFERRED EMBODIMENT

A vertebral arch spacer according to the invention will be described below in detail based on a preferred embodiment with reference to the accompanying drawings.

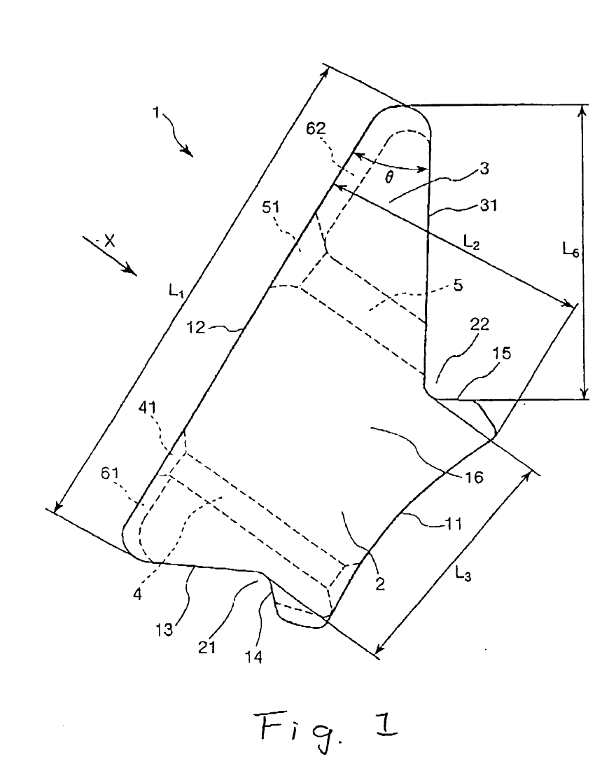

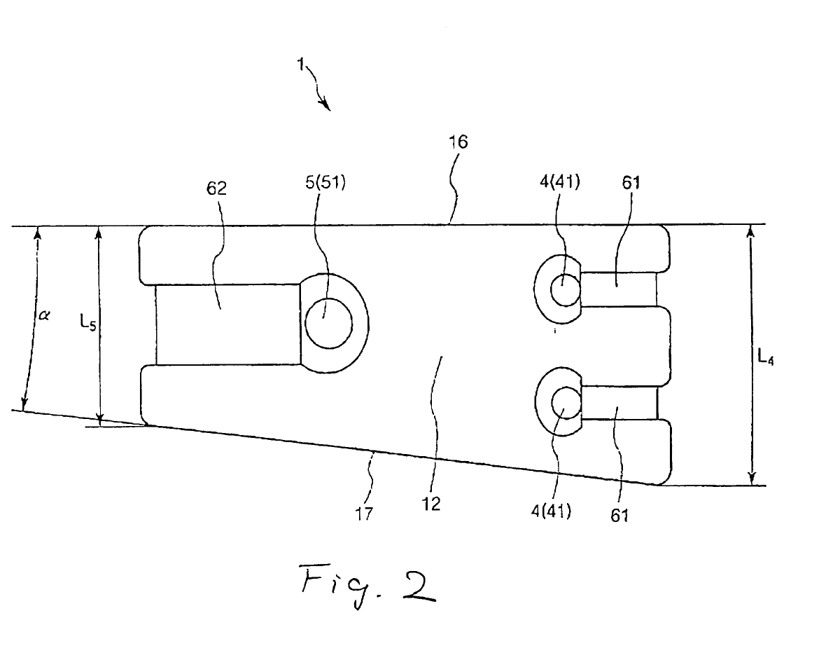

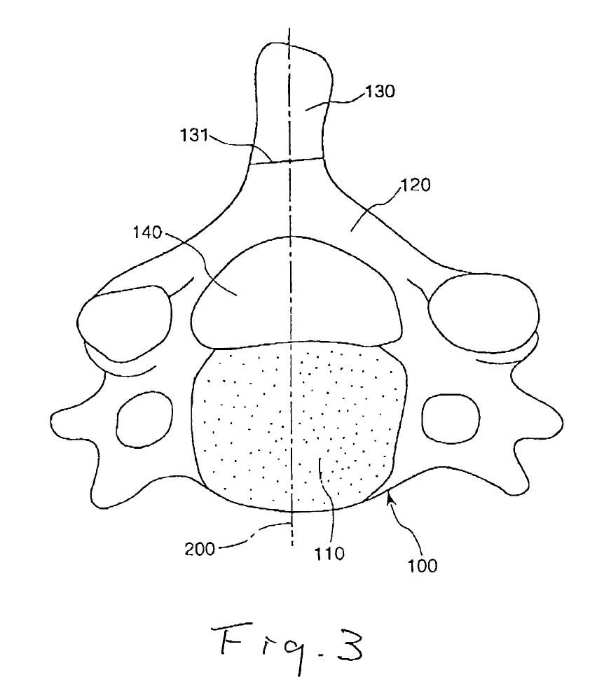

FIG. 1 is a plan view showing an embodiment of the vertebral arch spacer according to the invention, FIG. 2 is a view seen in a direction of an arrow X in FIG. 1, and FIGS. 3 to 6 are views for sequentially explaining a one-sided insertion, open-door type laminoplastic laminotomy using the vertebral arch spacer shown in FIG. 1, respectively.

A vertebral arch spacer 1 shown in FIGS. 1 and 2 is used in the one-sided insertion, open-door type laminoplastic laminotomy.

First of all, the one-sided insertion, open-door type laminoplastic laminotomy will be described with reference to FIGS. 3 to 6. In FIGS. 3 to 6, the upper side is set to be the back side (posterior part) and the lower side is set to be the abdomen side (anterior part).

[1] As shown in FIG. 3, a vertebra 100 of a cervical vertebra has a vertebral body 110, a vertebral arch 120 extended rearward from the vertebral body 110 (the upper side in FIG. 3) and surrounding a vertebral canal 140 (a vertebral foramen), and a spinous process 130 projecting rearward from the central part of the vertebral arch 120.

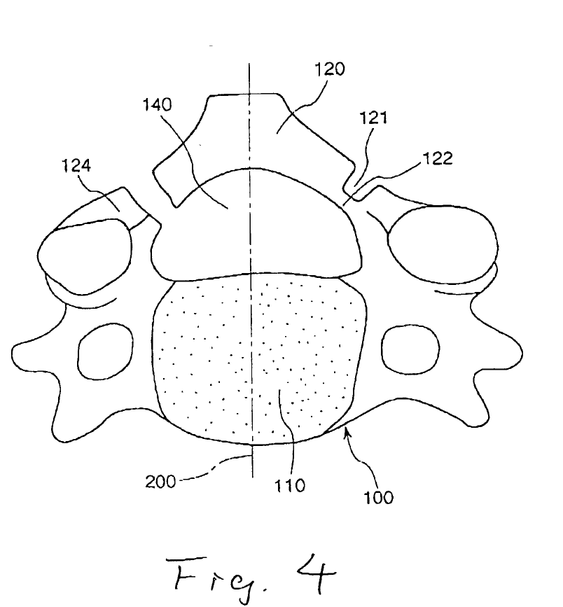

The spinous process 130 in the vertebral body 110 is separated (cut away) from the vertebral arch 120 along a cutting line 131 with a nuchal ligament, on-spine and inter-spine ligaments and a musculus (not shown) remaining attached thereto.

[2] As shown in FIG. 4, one of the sides of the outside portion of the vertebral arch 120 (the left side in FIG. 4) is cut by using an air drill, for example.

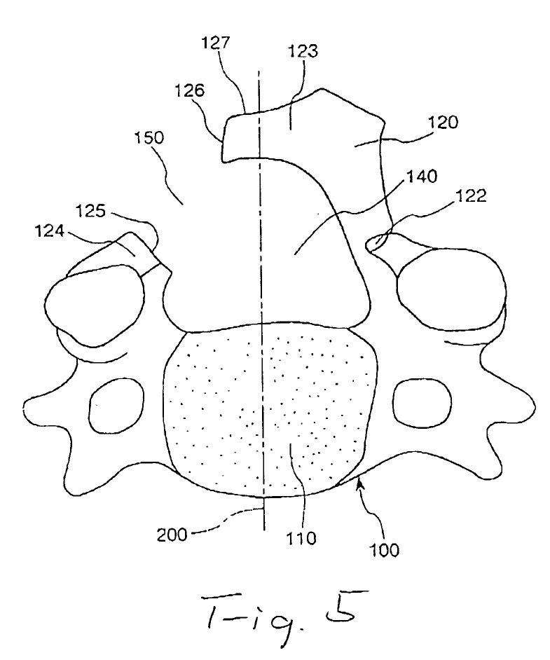

Moreover, a groove 121 is formed on the other side (the right side in FIG. 4) of the outside portion of the vertebral arch 120 by using the air drill, for example. The groove 121 has such a depth that only an outside plate is cut off and an inside plate is not cut off. The portion in which the groove 121 is formed acts as a hinge portion 122.

[3] As shown in FIG. 5, the vertebral arch 120 is opened outward (rotated) so as to be bent through the hinge portion 122. Consequently, a gap (a defective bone portion) 150 is formed between an opened portion 123 of the vertebral arch 120 and a portion 124 remaining on the vertebra side.

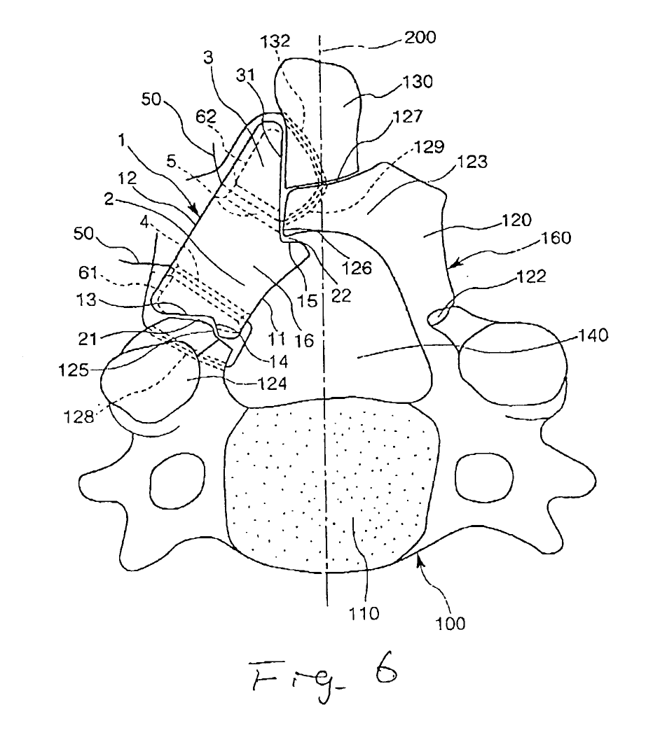

A cut portion (cut surface) 125 of the portion 124 remaining on the vertebra side of the vertebral arch 120 and a cut portion (cut surface) 126 of the opened portion 123 are reshaped if necessary.

[4] As shown in FIG. 6, the vertebral arch spacer 1 is inserted in the gap 150. Consequently, an expanded vertebral arch 160 is formed by the vertebral arch 120 of a patient and an insertion portion 2 of the vertebral arch spacer 1. The spinous process 130 cut away in the [1] is returned to a central (median) part and is caused to be adhered onto the extended vertebral arch 160 (the opened portion 123) by synostosis.

The vertebral arch spacer 1 is used in the one-sided insertion, open-door type laminoplastic laminotomy as described above. The structure of the vertebral arch spacer 1 will be described below.

In the following description, a direction is specified on the basis of a state in which the vertebral arch spacer 1 is inserted in (attached to) the operated portion (the gap portion 150) of the patient unless a specific definition is given. More specifically, a direction of the abdomen side (anterior part) of the patient (the lower side in FIGS. 1 and 6) will be referred to as “front”, a direction of the back side (posterior part) of the patient (the upper side in FIGS. 1 and 6) will be referred to as “rear”. Further, a direction of the head side of the patient (the upper side in FIG. 2) will be referred to as “upper” and a direction of the leg side of the patient (the lower side in FIG. 2) will be referred to as “lower”.

As shown in FIGS. 1 and 6, the vertebral arch spacer 1 has the insertion portion 2 to be inserted in the gap 150, and an abutment portion 3 capable of abutting on the spinous process 130 cut away from the vertebral arch 120.

As seen on a plane shown in FIG. 1, moreover, the vertebral arch spacer 1 mainly has such a shape as to be surrounded by a first surface 11, a second surface 12, a third surface 13, a fourth surface 14, a fifth surface 15 and an abutment surface 31.

As shown in FIG. 6, the first surface 11 faces the inside of the expanded vertebral arch 160 (the vertebral canal 140) in a state in which the insertion portion 2 is inserted in the gap 150, and the second surface 12 faces the outside of the expanded vertebral arch 160 in that state.

The first surface 11 and the second surface 12 are substantially in parallel with each other, and are inclined with respect to a median plane 200 to approach the median plane 200 rearward.

The first surface 11 is a curved concave surface. Consequently, the vertebral canal 140 can be expanded more largely (widely).

Each of the second surface 12, the third surface 13, the fourth surface 14, the fifth surface 15 and the abutment surface 31 is provided as a substantially planar surface.

As shown in FIG. 1, the third surface 13 is provided to form an acute angle with a portion on the front side of the second surface 12. The fourth surface 14 is provided to form an acute angle with a portion on the front side of the first surface 11. Moreover, the third surface 13 is formed to be longer than the fourth surface 14.

The third surface 13 and the fourth surface 14 form an obtuse angle. Consequently, a concave portion (a notched portion) 21 is formed.

The abutment surface 31 is provided to form an acute angle with a portion on the rear side of the second surface 12. The fifth surface 15 is provided to form an acute angle with a portion on the rear side of the first surface 11. Moreover, the abutment surface 31 is formed to be longer than the fifth surface 15.

The abutment surface 31 and the fifth surface 15 form an obtuse angle. Consequently, a concave portion (a notched portion) 22 is formed.

As shown in FIG. 6, the cut portion 125 of the portion 124 remaining on the vertebra side of the vertebral arch 120 abuts on (or approaches) the third surface 13 and the fourth surface 14. More specifically, at least a part of the cut portion 125 is inserted in the concave portion 21. Consequently, the cut portion 125 and the concave portion 21 are engaged with each other so that the shift of the vertebral arch spacer 1 can be prevented more reliably.

Moreover, the cut portion 126 of the opened portion 123 of the vertebral arch 120 abuts on (or approaches) the fifth surface 15 and a portion on the front side of the abutment surface 31. More specifically, at least a part of the cut portion 126 is inserted in the concave portion 22. Consequently, the cut portion 126 and the concave portion 22 are engaged with each other so that the shift of the vertebral arch spacer 1 can be prevented more reliably.

In the embodiment, thus, the concave portions 21 and 22 constitute engagement portions to be engaged with the cut portions 125 and 126, respectively.

As shown in FIG. 1, in the vertebral arch spacer 1, the insertion portion 2 is mainly surrounded by the first surface 11, the portion on the front side of the second surface 12, the third surface 13, the fourth surface 14, the fifth surface 15, and a portion on the front side of the abutment surface 31. The concave portions 21 and 22 are positioned on both ends of the insertion portion 2, respectively.

Moreover, the abutment portion 3 is mainly surrounded by the portion on the rear side of the second surface 12 and a portion on the rear side of the abutment surface 31.

As shown in FIG. 6, the abutment portion 3 is formed to be protruded outward from the insertion portion 2 (the outside of the expanded vertebral arch 160). Moreover, the abutment portion 3 is protruded rearward from the expanded vertebral arch 160.

More specifically, the abutment surface 31 in the abutment portion 3 is extended rearward beyond the opened portion 123 of the vertebral arch 120. Moreover, the abutment surface 31 is provided substantially in parallel with the median plane 200.

According to the vertebral arch spacer 1 of the invention, such an abutment portion 3 is provided so that the abutment surface 31 abuts on (or approaches) the cut-away spinous process 130 laterally (the left side in FIG. 6). More specifically, the spinous process 130 abuts on (or approaches) an external surface 127 of the opened portion 123 of the vertebral arch 120 and the abutment surface 31. Consequently, the position of the cut-away spinous process 130 can be held reliably.

By using the vertebral arch spacer 1 according to the invention, it is possible to reliably prevent the shift of the spinous process 130 even in the case in which the vertebrae are bent and extended after surgery, for example. Moreover, since the position of the spinous process 130 can be held stably, the spinous process 130 can be adhered to the opened portion 123 of the spinal arch 120 and the vertebral arch spacer 1 comparatively early by synostosis, and a physiological reconstruction can be thus carried out in the early stage. Moreover, it is also possible to shorten an external fixation period and a motion restriction period after the operation.

A length (L₆ in FIG. 1) of the abutment surface 31 is not particularly restricted and is preferably approximately 5 to 20 mm, and more preferably approximately 10 to 15 mm. If the L₆ is set within the range, the length of a portion of the abutment surface 31 to abut on (or to approach) the spinous process 130 can be maintained sufficiently. Consequently, the above-mentioned effect can be produced more remarkably. Moreover, the size of the vertebral arch spacer 1 is not increased unnecessarily.

If the L₆ is too small, the length of the portion of the abutment surface 31 to abut on (or to approach) the spinous process 130 may be insufficient, although it depends on a case or the position of the cutting line 131.

While the abutment surface 31 is formed as the substantially planar surface in the embodiment, it may not be planar. In other words, the abutment surface 31 can be a curved surface, for example, or can take another optional shape in order to cause an abutment state on the spinous process 130 and the cut portion 126 to be more excellent.

In order to prevent the slip of the spinous process 130 more reliably, moreover, it is also possible to provide a minute concavo-convex portion or groove on the abutment surface 31.

As shown in FIG. 2, an upper surface 16 and a lower surface 17 in the vertebral arch spacer 1 are formed as substantially planar surfaces, respectively. Moreover, the upper surface 16 and the lower surface 17 approach each other from the front side (the right side in FIG. 2) toward the rear side (the left side in FIG. 2). More specifically, the thickness of the vertebral arch spacer 1 (a dimension in a vertical direction) is gradually decreased from the front side toward the rear side (L₄>L₅ in FIG. 2).

Even if the cervical vertebra is bent (curved) rearward after surgery and a space with the same vertebral arch spacers 1 provided on upper and lower vertebrae (vertebral arches) is reduced, consequently, it is possible to more reliably prevent the vertebral arch spacers 1 from coming in contact with (interfering with) each other.

While an angle α (see FIG. 2) formed by the upper surface 16 and the lower surface 17 is not particularly restricted, it is preferably approximately 0 to 30 degrees and more preferably approximately 5 to 15 degrees in order to more effectively produce such an effect.

While a maximum thickness (L₄ in FIG. 2) of the vertebral arch spacer 1 is not particularly restricted, it is preferably approximately 8 to 15 mm. While a minimum thickness (L₅ in FIG. 2) is not particularly restricted, it is preferably approximately 5 to 12 mm.

In the illustrated structure, the thickness is gradually decreased from the front side toward the rear side over substantially the whole length of the vertebral arch spacer 1. The effect described above can be obtained even if the thickness is gradually decreased from the front side toward the rear side in a part of the whole length.

In the vertebral arch spacer 1, dimensions such as a length (L₁ in FIG. 1) of the second surface 12, a (maximum) distance (L₂ in FIG. 1) between the first surface 11 and the second surface 12, a distance (L₃ in FIG. 1) between the concave portion 21 and the concave portion 22, a length (L₆ in FIG. 1) of the abutment surface 31, and an angle (θ in FIG. 1) formed by the second surface 12 and the abutment surface 31 are appropriately determined depending on a case.

While a value of L₁/L₃is not particularly restricted, moreover, it is preferably approximately 1.1 to 3 and more preferably approximately 1.3 to 2.8.

In the illustrated structure, furthermore, the second surface 12, the third surface 13, the fourth surface 14, the fifth surface 15, the upper surface 16 and the lower surface 17 are formed as substantially planar surfaces, respectively, and do not need to be planar.

As shown in FIGS. 1 and 2, the vertebral arch spacer 1 is provided with two through holes 4 penetrating from the portion on the front side of the first surface 11 to the portion on the front side of the second surface 12. The two through holes 4 are provided in upper and lower parts.

Moreover, the vertebral arch spacer 1 is provided with a through hole 5 for penetrating from the portion on the front side of the abutment surface 31 to the portion on the rear side of the second surface 12.

A thread 50 serving as a fixing member can be inserted through each of the through holes 4 and 5.

As shown in FIG. 6, a through hole 128 is formed on the portion 124 remaining on the vertebra side of the vertebral arch 120, and the thread 50 is inserted through the through hole 4 and the through hole 128 and is bound (sutured). Thus, the portion 124 remaining on the vertebra side of the vertebral arch 120 and the vertebral arch spacer 1 can be fixed to each other. Consequently, it is possible to more reliably prevent the shift of the vertebral arch spacer 1 after surgery.

Moreover, a through hole 129 is formed on the opened portion 123 of the vertebral arch 120, and the thread 50 is inserted through the through hole 5 and the through hole 129 and is bound. Thus, the opened portion 123 of the vertebral arch 120 and the vertebral arch spacer 1 can be fixed to each other. Consequently, it is possible to more reliably prevent the shift of the vertebral arch spacer 1 after surgery.

Furthermore, a through hole 132 is formed on the spinous process 130 and the thread 50 inserted through the through hole 5 and the through hole 129 is also inserted through the through hole 132 so that the spinous process 130 can be fixed. Consequently, the position of the spinous process 130 can be held more reliably so that the shift of the spinous process 130 can be prevented more reliably.

It is apparent that the numbers of the through holes 4 and 5 are not restricted to those in the illustrated structure. Moreover, the fixing member is not restricted to the thread 50 but may be another linear member such as a wire or a bolt.

As shown in FIGS. 1 and 2, a groove 61 capable of inserting the thread 50 therein is formed in a portion from the edge portion of an opening 41 of the through hole 4 provided on the second surface 12 to the corner portion between the second surface 12 and the third surface 13. Moreover, a groove 62 capable of inserting the thread 50 therein is formed in a portion from the edge portion of an opening 51 of the through hole 5 provided on the second surface 12 to the corner portion between the second surface 12 and the abutment surface 31.

Each thread 50 is further bound with a tension in the state shown in FIG. 6 and is then inserted into each of the grooves 61 and 62. Consequently, the shift and looseness of the thread 50 can be prevented and the fixation can be maintained more reliably by the thread 50.

It is preferable that the vertebral arch spacer 1 should be constituted by a ceramics material. Since the ceramics material has an excellent workability, a shape and size thereof can easily be regulated by cutting using a lathe or a drill.

Various ceramics materials can be used, and bioceramics such as alumina, zirconia and a calcium phosphate based compound are particularly preferable. In particular, since the calcium phosphate based compound has an excellent bioaffinity, it is especially preferable as the constitutive material of the vertebral arch spacer 1.

Examples of the calcium phosphate based compound include apatites such as hydroxyapatite, fluoroapatite and apatite carbonate, calcium diphosphate, calcium triphosphate, calcium tetraphosphate and calcium octaphosphate, and one of them or more can be mixed for use. Moreover, the calcium phosphate based compound having a Ca/P ratio of 1.0 to 2.0 is preferably used.

In the calcium phosphate based compounds, the hydroxyapatite is more preferable. Since the structure of the hydroxyapatite is the same as that of the inorganic principal component of a bone, the hydroxyapatite has an excellent biofitness. When the vertebral arch spacer 1 is to be manufactured, it is more preferable that a hydroxyapatite particle to be a raw material should be provisionally burned at 500 to 1000° C. The hydroxyapatite particle burned provisionally at such a temperature has an activity controlled to some extent. Therefore, a sintering unevenness can be prevented from being caused by the rapid progress of sintering so that a sintered product having no strength unevenness can be obtained.

In the invention, the porosity of the ceramics is preferably 0 to 70% and more preferably 30 to 50%. By setting the porosity within this range, it is possible to obtain an excellent bioaffinity while maintaining a strength, thereby promoting a bone neoplasm by a bond conduction.

For the constitutive material of the vertebral arch spacer 1 according to the invention, it is also possible to use a composite material of the ceramics material and a metal material having a small biodamaging property such as titanium in addition to the ceramics material.

While the vertebral arch spacer according to the invention has been described above based on the embodiment shown in the drawing, the invention is not restricted thereto but each portion constituting the vertebral arch spacer can be replaced with an optional structure capable of fulfilling the same functions.

Moreover, it is apparent that the invention can also be applied to a vertebral arch spacer to be used when a vertebral arch is to be opened in the opposite direction to that shown in the drawing. Furthermore, the vertebral arch spacer according to the invention can be used on any of the left and right sides (by turn-over).

### EXAMPLE

A hydroxyapatite slurry (Ca/P ratio=1.67) was prepared by a well-known wet synthetic method from a calcium hydroxide slurry and a phosphoric acid water solution. This was dried by a spray thermal drying method and was then burned provisionally at 700° C. in an atmospheric furnace so that spherical powder was obtained.

Next, the spherical powder of the hydroxyapatite thus obtained and a high molecular compound water solution were mixed and stirred, and the mixture was then dried so that a hydroxyapatite block product was obtained.

A contraction after sintering was calculated from the block product, and a molded product having the shape of a desirable vertebral arch spacer was fabricated by using a lathe or a drill.

The molded product was put in an electric furnace and was sintered for 4 hours at 1200° C. Thus, a vertebral arch spacer having the shape shown in FIGS. 1 and 2 was fabricated.

Each portion of the vertebral arch spacer according to the embodiment had the following dimension, that is, the length L₁ of the second surface 12: 18 mm, the maximum distance L₂ between the first surface 11 and the second surface 12: 11 mm, the distance L₃ between the concave portion 21 and the concave portion 22: 8 mm, the maximum thickness L₄: 10 mm, the minimum thickness L₅: 6 mm, the length L₆ of the abutment surface 31: 11 mm, and the angle θ formed by the second surface 12 and the abutment surface 31: 25 degrees. Moreover, the porosity of the hydroxyapatite was set to be 40%.

By using the vertebral arch spacer, the one-sided insertion, open-door type laminoplastic laminotomy was carried out by the above-mentioned method for the cases of ten patients having a vertebral canal stenosis.

As a result, a stenosed vertebral canal was expanded to have an approximate shape to a normal vertebral canal for all the patients. Moreover, the spinous process cut away can be fixed easily.

The progress after surgery was well and the shift of the spinous process and curvature deformation were not caused even if a long period of time passed after surgery. The shifts of the vertebral arch and the vertebral arch spacer were not confirmed and the vertebral canal was maintained in a good expanded state. Moreover, the vertebral arch spacer, the vertebral arch and the spinous process were synostosis-bonded quickly and a physiological reconstruction could be carried out very well. Furthermore, the stable reconstruction could be carried out so that an external fixation period and a motion restriction period could be shortened.

[Effect of the Invention]

As described above, according to the invention, it is possible to fill up the gap formed by opening the vertebral arch toward either side, thereby reliably extending the vertebral arch.

Moreover, the abutment portion capable of abutting on the spinous process cut away from the vertebral arch is provided. Consequently, the shift of the spinous process can be prevented and the synostosis can be carried out in the early stage.

From the foregoing, the physiological reconstruction can be carried out well in the early stage, and the external fixation period and the motion restriction period can also be shortened.

What is claimed is:

1. A vertebral arch spacer to be inserted into a gap formed by cutting one of sides of a vertebral arch and opening the vertebral arch by using the other side as a hinge portion, comprising:
an insertion portion to be inserted in the gap; and
an abutment portion capable of abutting on a spinous process cut away from the vertebral arch.

2. The vertebral arch spacer according to claim 1, wherein the abutment portion is formed to be protruded outward from the insertion portion.

3. The vertebral arch spacer according to claim 1, wherein the abutment portion is protruded rearward from the expanded vertebral arch when the insertion portion is inserted in the gap.

4. The vertebral arch spacer according to claim 1, wherein the abutment portion has an abutment surface to be substantially parallel with a median plane when the insertion portion is inserted in the gap.

5. The vertebral arch spacer according to claim 1, wherein the abutment portion can abut on the spinous process laterally.

6. The vertebral arch spacer according to claim 1, further comprising a first surface facing an inside of the expanded vertebral arch and a second surface facing an outside of the expanded vertebral arch when the insertion portion is inserted in the gap.

7. The vertebral arch spacer according to claim 6, wherein the first surface is a curved concave surface.

8. The vertebral arch spacer according to claim 6, wherein the abutment surface capable of abutting on the spinous process and the second surface form an acute angle.

9. The vertebral arch spacer according to claim 1, further comprising a through hole capable of inserting a fixing member to be used for a fixation to an opened portion of the vertebral arch.

10. The vertebral arch spacer according to claim 9, wherein the spinous process can be fixed by inserting the fixing member through a through hole formed on the spinous process.

11. The vertebral arch spacer according to claim 1, further comprising a through hole capable of inserting a fixing member to be used for a fixation to a portion remaining on a vertebral body side of the vertebral arch.

12. The vertebral arch spacer according to claim 9, further comprising a groove in which the fixing member can be inserted.

13. The vertebral arch spacer according to claim 1, wherein an engagement portion capable of being engaged with a cut portion of the vertebral arch is provided on each of ends of the insertion portion.

14. The vertebral arch spacer according to claim 13, wherein the engagement portion is constituted by a concave portion or a notched portion in which at least a part of the cut portion can be inserted.

15. The vertebral arch spacer according to claim 1, further comprising a portion in which a thickness is gradually decreased from a front side toward a rear side.

16. The vertebral arch spacer according to claim 1, wherein a ceramics material is used as a constitutive material.

17. The vertebral arch spacer according to claim 16, wherein the ceramics material comprises a calcium phosphate based compound.

18. The vertebral arch spacer according to claim 17, wherein the calcium phosphate based compound has a Ca/P ratio of 1.0 to 2.0.

19. The vertebral arch spacer according to claim 17, wherein the calcium phosphate based compound is hydroxyapatite.

20. The vertebral arch spacer according to claim 1, wherein a porosity of the vertebral arch spacer is 0 to 70%.
